(19) United States
(12) Patent Application Publication
(10) Pub. No.: US 2016/0058601 A1
(43) Pub. Date: Mar. 3, 2016

(54) ADJUSTABLE COLLAR

(71) Applicant: Aspen Medical Partners, LLC, Irvine, CA (US)

(72) Inventors: Geoffrey Garth, Long Beach, CA (US); Steven Burke, Huntington Beach, CA (US); Albert Romo, Lakewood, CA (US); Jozsef Horvath, Fullerton, CA (US)

(21) Appl. No.: 14/846,609

(22) Filed: Sep. 4, 2015

Related U.S. Application Data
(63) Continuation-in-part of application No. 14/638,519, filed on Mar. 4, 2015.
(60) Provisional application No. 61/947,676, filed on Mar. 4, 2014.

(51) Int. Cl. A61F 5/055

(52) U.S. Cl. CPC A61F 5/055

(57) ABSTRACT

Adjustable braces providing improved levels of adjustability and ease of use are provided. Two components of an adjustable brace can be pivotally fastened to one another at two ends, and define a size-adjustable opening therebetween. The two components can be coupled to one another via an adjustment mechanism that allows for an adjustment of a vertical position of one component relative to another component. Two pivoting locking member can be provided that captures an adjustment and blocks a movement of the adjustment mechanism.

This application is a continuation-in-part of, and claims priority to, U.S. patent application Ser. No. 14/638,519 filed Mar. 4, 2015, which claims priority to U.S. provisional patent application No. 61/947,676 filed Mar. 4, 2014. These and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

### FIELD OF THE INVENTION

The field of the invention is orthosis, including adjustable collars.

### BACKGROUND

The following description includes information that may be useful in understanding the present invention. It is not an admission that any of the information provided herein is prior art or relevant to the presently claimed invention, or that any publication specifically or implicitly referenced is prior art.

Cervical collars are often used to maintain a spine in neutral alignment by supporting a user's chin at a particular position. Cervical collars can be supplied for temporary or long-term use, and emergency personnel typically have cervical collars readily available for use on patients having traumatic head or neck injuries. Because potential users of collars are sized and proportioned differently, and storage of bulky collars can be burdensome, many have attempted to provide one-size-fits-most adjustable collars that can be used by different users having different or varying heights, weights and proportions.

For example, U.S. Pat. No. 5,795,315 to Traut teaches a generally flat neck encircling band that can be sized to a standard size prior to being worn, and a mandible support that couples to the band via slots, sliders, locking holes and locking tabs such that the mandible support can be positioned at one of a plurality of vertical positions relative to the band.

Unfortunately, the Traut collar apparently cannot be adjusted when worn, does not allow a user to simultaneously adjust left and right sides, requires separate adjustment and locking members to be located at opposing extremeties of the mandible support, and suffers from various other disadvantages.

Thus, there is still a need for improved adjustable collars.

### SUMMARY

The inventive subject matter provides apparatus, systems and methods in which two components of a brace can be coupled to one another in a manner that allows for an adjustment and secured positioning of one component relative to the other via a simple manipulation of one or more adjustment pieces.

In some aspects, a brace comprises a chin support coupled to a collar body, an adjustment mechanism that enables an adjustment of a vertical position of the chin support relative to the collar body, and one or more pivotable, slidable, retractable, expandable, or otherwise movable locking panels.

Some contemplated adjustment mechanisms include at least two adjustment ends that extend below the chin support. The adjustment ends could comprise two ends of a single adjustment piece, or could comprise two separate adjustment pieces. Additionally or alternatively, the adjustment ends (or adjustment piece) could be considered a part of the chin support, or separate components (or separate component) that are coupled to the chin support.

Two notched portions could be included on left and right sides of the collar body to align with at least portions of the adjustment ends. Alternatively, the collar body could be coupled with at least two notched members, each notched member including a plurality of notches (e.g., 2, 3, 4, 5, or even more notches).

Each notch of a notched member could be sized and dimensioned to receive at least a portion of an adjustment end's member or protrusion. When a member or protrusion of an adjustment end is positioned within a notch (at least partially received by the notch), the adjustment end could be considered to be in a lockable configuration. Where the at least two adjustment ends are part of a single adjustment piece, the adjustment piece could be considered to be in a lockable configuration when each adjustment end's member or protrusion is positioned within a notch. When a member or protrusion of an adjustment end is not positioned within a notch, and thus can be moved between lower or higher notch positions, the adjustment end could be considered to be in a movable or adjustable configuration.

A movable locking panel could be pivotally, rotatably, retractably, extendably or slidably coupled with the collar body or notched member, and include a receiving end that is sized and dimensioned to wrap around an outer edge of a notched member and at least a portion of an adjustment end when the adjustment end is in a lockable configuration.

In some aspects, the locking panel may not be able to wrap around both the outer edge of the notched member and the portion of the adjustment end when the adjustment end is in a movable configuration, at least not without damage or breakage. Viewed from another perspective, the panel could be positioned on the collar body, and sized and dimensioned to lock an adjustment end in place when the adjustment end is in a lockable configuration, but to not allow locking of the adjustment end when the adjustment end is in a movable configuration.

It is contemplated that the adjustment end could include an outer ridge that an internal ridge of the panel is sized and dimensioned to overlap. Additionally or alternatively, the receiving end of the panel could include an outer wall, a front wall and a narrow inner wall (e.g., less than 20 mm wide, less than 15 mm wide, less than 10 mm wide, or even less than 5 mm wide). In some aspects, the distance between the outer and inner walls could be substantially the same as (e.g., within 15%, within 10%, or even within 5% of) the thickness of an adjustment end. In some aspects, the adjustment end could include a vertically oriented ridge, and the distance between the outer and inner walls could be substantially the same as (e.g., within 15%, within 10%, or even within 5% of) the distance between an edge of the adjustment end and the vertically oriented ridge. In such embodiments, the panel could be in a locked position when its inner wall overlaps with the vertically oriented ridge to lock the adjustment end in place in a locked configuration.

To release the locking panel from a locked position, a user could pull, push or otherwise manipulate a release tab coupled to the inner wall of the panel (or any other portion of the panel, for example, an outer wall, a front wall) to overcome the overlap of the panel's inner wall with the adjustment end or vertically oriented ridge.

Another contemplated collar of the inventive subject matter includes a chin support adjustably coupled to a collar body. The chin support could include or be coupled to one or more adjustment ends, and each of the adjustment ends could comprise a member and a movable locking mechanism. The collar body could include or be coupled to one or more notched members, and each notched member could comprise at least two notches with an open outer edge and a recessed portion.

Some members are rod-like, horizontally oriented, and include inner and outer portions having different thicknesses. As used herein the inner portion is the portion closer to a vertical midline of the wearer when the collar is worn. Additionally, some notches could be oriented such that the open edge to recessed portion extends orthogonally to the member. The open edge could be sized and dimensioned to receive the thinner portion, and be too small to receive the thicker portion. In such embodiments, the member could be secured within a notch by entering the open edge via the thinner portion, and being released such that the thicker portion at least partially rests within the recessed portion. The secured position can be maintained by at least one of the following: (1) the adjustment end could be biased in the secured position such that physical force is required to move the thinner portion to align with the open edge; and (2) a locking member blocks a horizontal movement of the member.

As used herein, “horizontally oriented” objects includes an objects that are exactly horizontal, as well as objects that are within 25 degrees of the horizontal (e.g., within 20 degrees, within 15 degrees, within 10 degrees, or within 5 degrees of the horizontal). Similarly, “vertically oriented” objects includes an objects that are exactly vertical, as well as objects that are within 25 degrees of the vertical (e.g., within 20 degrees, within 15 degrees, within 10 degrees, or within 5 degrees of the vertical). As used herein, notches (or other object) that “extend orthogonally” or “extend perpendicularly” to an object includes notches that are exactly perpendicular to an object, as well as notches that form an angle of between 65 and 115 degrees with the object. Therefore, it should be appreciated that horizontally objects and objects that extend perpendicularly to another object do not need to be straight as long as they fall within the ranges described herein.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

In some aspects of the inventive subject matter, a curved chin support can be pivotally or otherwise adjustably attached to a curved collar body on right and left sides to form a size-adjustable tracheal opening. The chin support could include a chin cup and a centrally located single adjustment piece coupled with the chin cup. The adjustment piece could include two end portions that are configured to couple with notched members of the collar body, the two end portions each including rod-like members. Some exemplary adjustment pieces include an upside-down U-shape, where the free end portions of the “U” are considered the two free ends (or adjustment ends) of the adjustment piece. It should be appreciated that a U-shape could comprise any two vertically oriented pieces connected by a central portion, the two vertically oriented pieces including the two free ends. Some other exemplary adjustment pieces include two adjustment ends that are separately coupled to the chin support.

The collar body could include left and right notched members, each including a plurality of vertically arranged notches. Each notched member could be positioned, sized and dimensioned to separately receive members of the left and right adjustment ends.

In some preferred embodiments, the adjustment piece or the adjustment ends could be biased. Where an open end of a notch is sized and dimensioned to receive any portion of an adjustment end's member, or both the thicker and thinner portion(s), it is contemplated that the adjustment piece or ends could be biased in the direction of the notch's recessed portion. In such embodiments, a user could adjust a size of the tracheal opening by moving the adjustment portion or ends forward and out through the open edge. In some contemplated embodiments, the ends could be biased to be positioned further away from one another horizontally, or could be biased to be positioned closer to one another horizontally. In such embodiments, the user could adjust a size of the tracheal opening by squeezing the ends towards one another (or separating the ends further away from one another), and then moving them forward and out through the open edges.

It should be appreciated that a member or rod could have any suitable cross-sectional shape (e.g., circle, oval, square, rectangle, triangle, pentagon, hexaton, diamond or irregular) and does not need to be circular or even uniform.

In other aspects, a rear support could be provided for fastening to the front portion of a brace (chin support and collar body). The rear support could include right and left side fasteners that are complementary to right and left side fasteners located on an outer or other surface of the front portion of the brace. Preferably the fasteners allow for adjustment of a circumference of the brace to accommodate necks of different sizes (e.g., hook and loop fasteners, snap fasteners).

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1A is a front perspective view of a brace of the inventive subject matter.

FIG. 1B is a left-side perspective view of the brace of FIG. 1A.

FIG. 2A is a side perspective view of the adjustment mechanism of the brace of FIGS. 1A-1B.

FIG. 2B illustrates a rear view of the U-shaped Adjustment Piece of FIG. 2A.

FIG. 3A is a side view of the brace of FIG. 1A-2B in a first configuration.

FIG. 3B is a side view of the brace of FIG. 1A-2B in a second configuration.

FIG. 3C is a side view of the brace of FIG. 1A-2B in a third configuration.

FIG. 3D is a side view of the brace of FIG. 1A-2B in a fourth configuration.

FIG. 4A is a front view of another brace of the inventive subject matter.

FIG. 4B is a side perspective view of the brace of FIG. 4A.

FIG. 5A is a top view of a chin support of the brace of FIG. 4A.

FIG. 5B is a top view of a first end of an adjustment piece of FIG. 5A.

FIG. 6A is a front view of the adjustment piece of FIG. 5A.

FIG. 6B is a front view of the adjustment piece of FIG. 5A being adjusted.

FIG. 7A is a side perspective view of the collar body of FIG. 4A.

FIG. 7B is a front top view of the adjustment piece of FIG. 5A.

FIG. 7C is a front, top perspective view of the adjustment piece of FIG. 5A when the adjustment piece is squeezed.

FIG. 7D is a rear, side perspective view of the brace of FIG. 4A showing a locking member in a locked position.

FIG. 8 is a side view of the brace of FIG. 4A showing a locking member in an open position.

FIG. 9A is a top view of another brace having an alternative embodiment of a locking member.

FIG. 9B is a side perspective view of the brace and locking member of FIG. 9A.

FIG. 10A is a front, side perspective view of yet another brace of the inventive subject matter having a pivotable locking panel.

FIG. 10B is a front view of the brace of FIG. 10A.

FIG. 10C is a side view of the brace of FIG. 10A.

FIG. 10D is a perspective view of the brace of FIG. 1 OA showing an adjustment end in a lockable configuration.

FIG. 10E is a perspective view of the brace of FIG. 10A showing an adjustment end in a movable configuration.

FIG. 10F is a perspective view of the brace of FIG. 10A showing locking panels in open positions.

FIG. 10G is a perspective view of the brace of FIG. 10A showing locking panels in locked positions.

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The inventive subject matter provides apparatus, systems and methods in which two components of a brace can be coupled to one another in a manner that allows for an adjustment and secured positioning of one component relative to the other via a simple manipulation of a single adjustment piece. Viewed from another perspective, the braces described herein allow the left and right sides of one component of a brace to be simultaneously adjusted relative to another component via at least one of (a) simultaneous manipulation of two adjustment ends, and (b) manipulation of a single centrally-located adjustment piece.

As used herein, the term “centrally-located” should be interpreted broadly to include an object whose midline is located at the midline of a second object or anywhere within 15% of the midline in either direction (e.g., up/down or left/right) relative to the entire length of the second object. In some preferred embodiments, a centrally-located adjustment piece will include a midline that is located within 5% of the midline, more preferably at the midline, of the chin support.

While the discussion herein is generally directed to cervical collars, one should appreciate that the adjustment mechanisms provided herein could be used with any commercially suitable orthotic device having at least two components that adjustably (e.g., pivotally, rotatably, slidably) coupled.

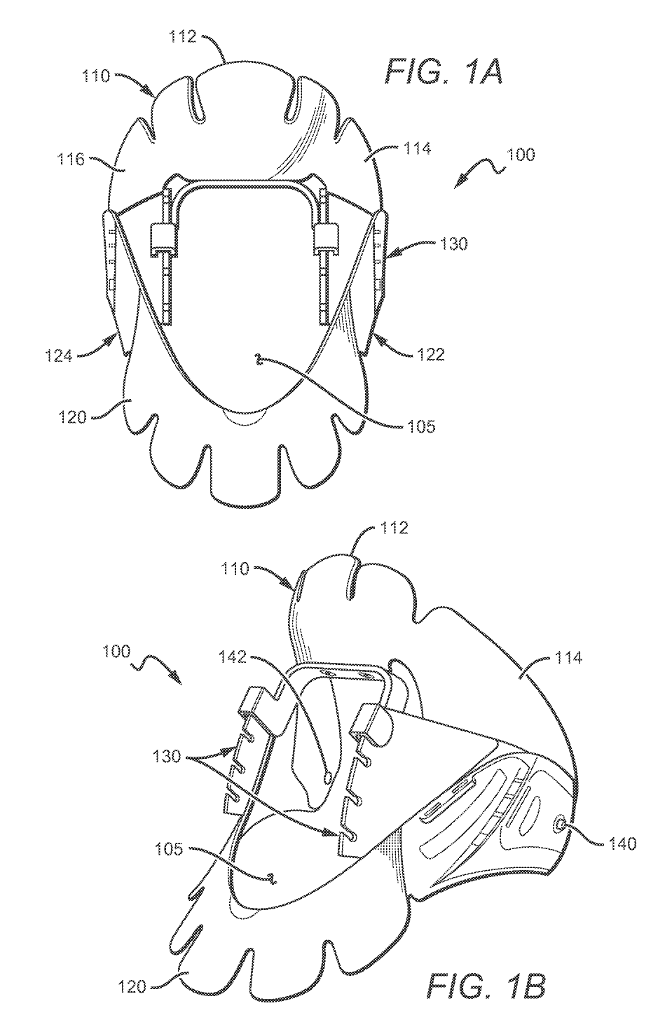

An exemplary adjustable collar of the inventive subject matter is shown in FIGS. 1A-1B. FIG. 1A illustrates a front perspective view of brace 100. FIG. 1B illustrates a left-side perspective view of brace 100. Brace 100 includes a chin support 110 and collar body 120, which are pivotally coupled to one another via fasteners (e.g., 140 and 142) and define a size-adjustable tracheal opening 105 that provides access for airway management and other procedures.

As used herein, the term “size-adjustable tracheal opening” refers to an opening of a brace that provides an access point to a wearer's trachea (e.g., for a tracheotomy) when the brace is worn, and is adjustable in size along at least one of a diameter, a length and a width.

Chin support 110 includes a chin cup 112 and laterally extending left and right side wings 114 and 116 located on opposite sides of the chin cup 112. The chin cup and laterally extending wings could be made of a single piece of material, or could comprise separate pieces of material that are coupled to one another. Collar body 120 includes a front portion (shown here as being toothed) and left and right side portions 122, 124, respectively. Similarly to the chin support, the front and side portions could be made of a single piece of material, or could comprise separate pieces of material that are coupled with one another. The two components (chin support 110 and collar body 120) are pivotally fastened to one another via a first fastener 140 that couples wing 114 and left side portion 122, and a second fastener 142 that couples wing 116 and right side portion 124.

While chin supports, collar bodies, locking members, locking panels, notched members, and adjustment ends of the inventive subject matter are preferably made at least in part of sturdy and stiff materials such as plastics, molded thermoplastics (e.g., polyethylene) or thermoset plastics such that each can permanently maintain a shape, it should be appreciated that the components of a brace could each be made of any suitable material or materials, including for example, a rubber, a metal, a foam, a flexible plastic, or a fabric. In some preferred braces, the inner surface of the chin support and collar body are coupled with padding material(s) via hook and loop or other fastener(s) such that the inner surfaces of the chin support and collar body are lined partially or entirely with a padding material to prevent or reduce discomfort to a wearer.

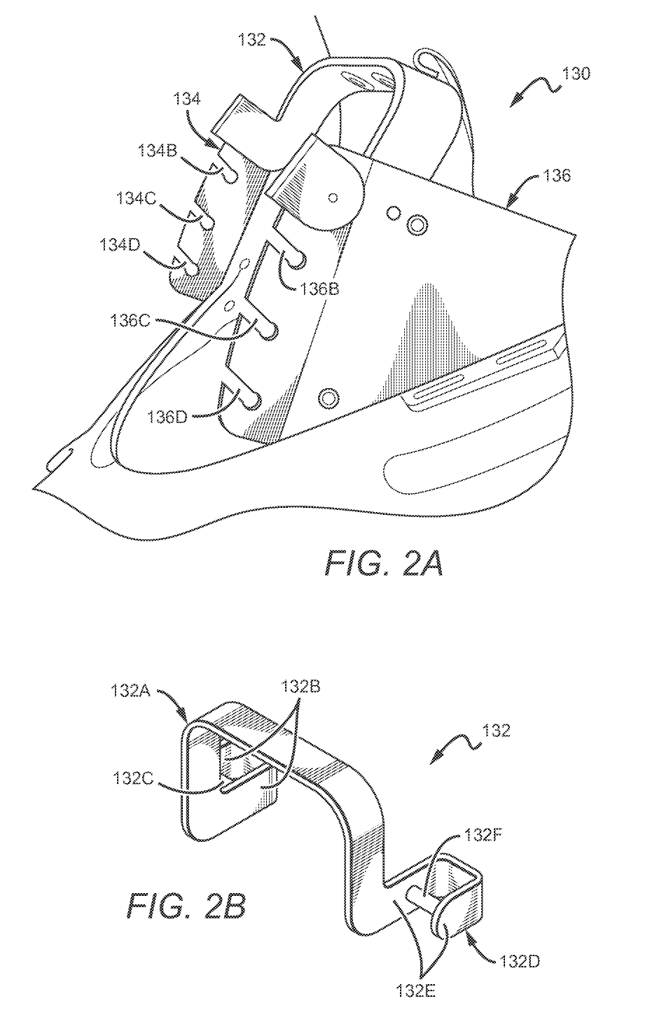

Brace 100 also includes an adjustment mechanism 130, which is shown in detail in FIGS. 2A-2B. Adjustment mechanism 130 advantageously allows a user (such as a wearer or a medical or emergency professional) to adjust a vertical position of chin support 110 relative to the collar body 120 via a single-handed manipulation of two adjustment ends, here the adjustment ends being part of a centrally located adjustment piece. Viewed from another perspective, adjustment mechanism 130 allows a user to adjust a size of a tracheal opening formed by the chin support and collar body via a single-handed manipulation of a centrally located adjustment piece.

As illustrated, adjustment mechanism 130 includes a U-shaped adjustment piece 132 that is coupled with chin cup 112. Adjustment piece 132 could be fixedly or movably coupled with the chin cup, for example, via one or more screws, hinges, or other fasteners. While some adjustment pieces are generally described herein as being U-shaped, it should be appreciated that adjustment piece could comprise any suitable shape that includes at least one attachment point and at least two adjustment ends that can couple with notched members of the collar body.

Where the adjustment piece 132 is fixedly attached to chin piece 112, it is contemplated that the adjustment piece 132 could be movable from an unengaged position to an engaged position via the flexibility of the chin cup 112. For example, the chin cup could be toothed such that the chin cup can more easily be manipulated. Where the adjustment piece 132 is movably attached to chin piece 112 (e.g., pivotally attached via a hinge and spring biasing mechanism), the adjustment piece 132 could be movable from an unengaged position to an engaged position without flexing or deforming of the chin cup 112.

U-shaped adjustment piece 132 includes a left end 132A and a right end 132D, each of which comprise a pair of parallel walls, 132B and 132E, respectively, and a member 132C and 132F, respectively, that extend between the parallel walls. Additionally, adjustment mechanism 130 includes a first notched member 134 and a second notched member 136 disposed on right and left sides of the collar body 120, respectively. While members 132C and 132F are shown to be disposed between parallel walls of the U-shaped adjustment piece, it is contemplated that the members could instead be located to project from the left and right ends 132A and 132D, respectively.

As used herein, the term “notched member” should be interpreted broadly to include members having two or more recesses, indentations or incisions along an edge, as well as members having one or more openings (through-holes) there-through. Similarly, the term “notch” should be interpreted broadly to include a recess, indentation or incision, as well as an opening. As one non-limiting example, a notched member could comprise a notched rail or slot including a vertical portion that guides a protrusion, projection or rod-like member to a plurality of substantially perpendicular portions (notches) where the protrusion, projection or rod-like member could rest.

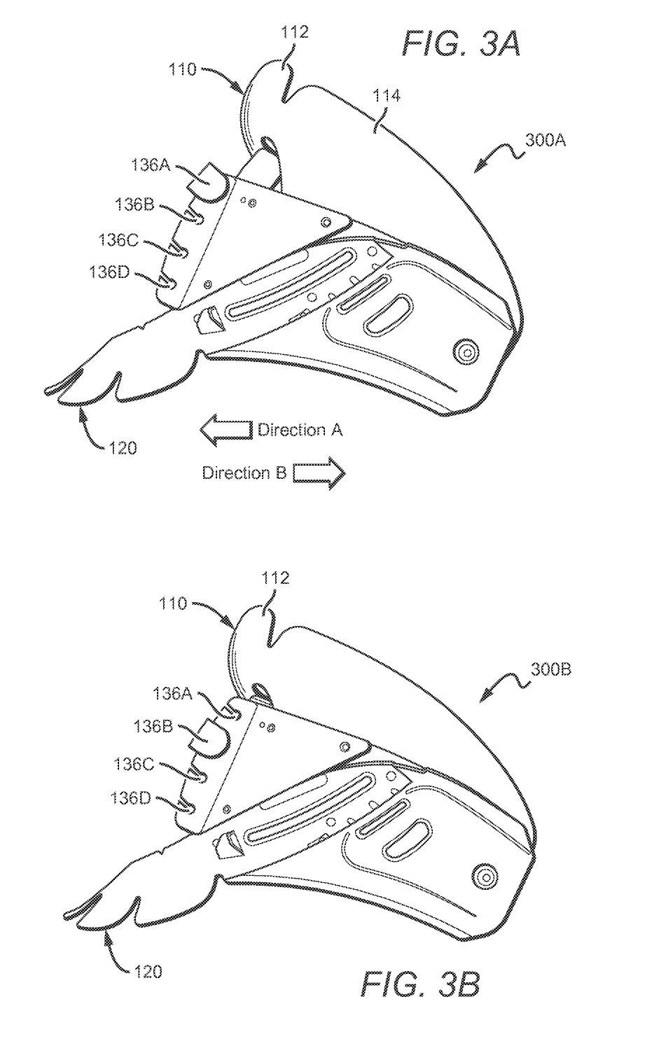

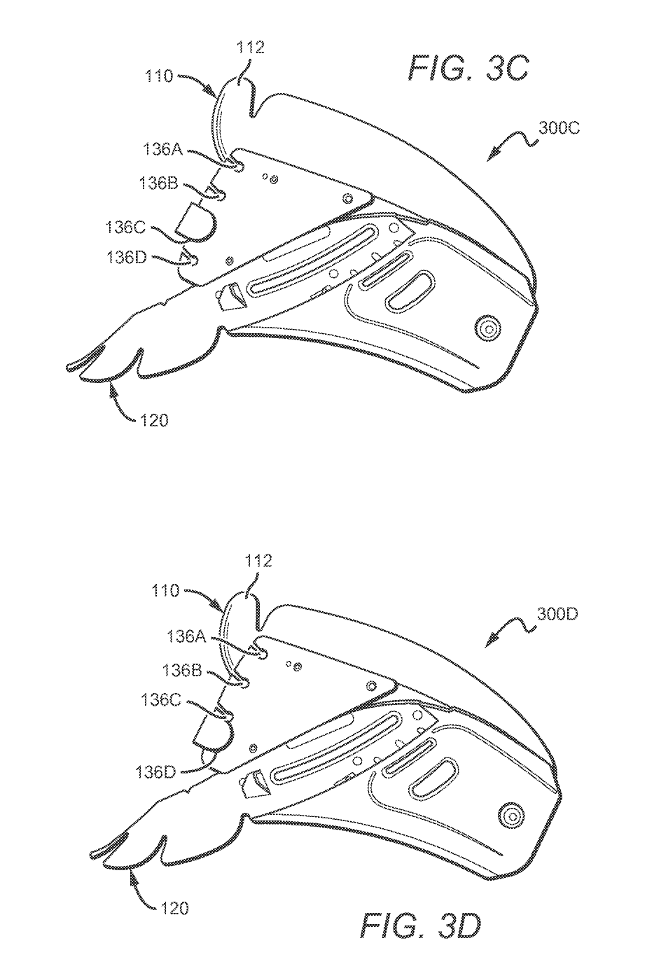

First notched member 134 includes a plurality of notches including first notch 134A (see FIGS. 3A-3D), second notch 134B, third notch 134C and fourth notch 134D. Second notched member 136 includes a plurality of notches including fifth notch 136A (see FIGS. 3A-3D), sixth notch 136B, seventh notch 137C and eighth notch 136D. Preferably each of the notches of the first and second notched members are horizontally aligned with at least one other notch (e.g., 134A-136A, 134B-136B, 134C-136C, 134D-136D), and sized and dimensioned to separately receive member 132C or 132F.

While brace 100 has an adjustment piece 132 that is disposed on the chin support 110 (e.g., coupled with chin piece 112) and notched members 134 and 136 disposed on the collar body 120, it should be appreciated that in some embodiments the adjustment piece could be disposed on the collar body while the notched members could be disposed on sides of the chin support such that the notches vertically align with the adjustment piece's left and right ends.

As illustrated in FIGS. 3A-3D, brace 100 can be adjusted to be in one of at least four configurations. It should be appreciated that a brace of the inventive subject matter could include any suitable number of notches (e.g., 2, 4, 6, 8, 10 or 12) based on a number of different configurations suitable for the brace. For example, where a brace is an adjustable collar and 6 different configurations (lengths between chin cup and collar body) are desired, each of two notched members could comprise 6 notches, totaling 12 notches.

In FIG. 3A, brace 100 is in configuration 300A, which provides the greatest vertical distance between chin cup 112 of chin support 110 and a fixed point (e.g., bottommost central point) of collar body 120. In configuration 300A, first notch 134A and fifth notch 136A are horizontally aligned, and left end member 132C and right end member 132F are similarly horizontally aligned. Additionally, left end member 132C is disposed partially or fully within fifth notch 136A and right end member 132F is disposed partially or fully within first notch 134A. U-shaped adjustment piece 132 is biased in Direction B and notches 134A and 136A can optionally be slanted down and inwards. In some embodiments, a locking mechanism may not be required to keep brace 100 in configuration 300A (or 300B, 300C, 300D), even while brace 100 is being worn.

In FIG. 3B, brace 100 is in configuration 300B, which provides for a shorter vertical distance between chin cup 112 and the fixed point of collar body 120 than configuration 300A. In configuration 300B, left end member 132C is disposed partially or fully within sixth notch 136B and right end member 132F is disposed partially or fully within second notch 134B, which is horizontally aligned with sixth notch 136B. Brace 100 could be adjusted from configuration 300A to 300B via a simple manipulation of adjustment piece 132 in Direction A, and release of adjustment piece 132 in Direction B once members 132C and 132F are aligned with notches 136B and 134B. Viewed from another perspective, manipulation of adjustment piece 132 simultaneously moves members 132C and 132F from a first pair of notches (134A and 136A) to a second pair of notches (134B and 136B).

In FIG. 3C, brace 100 is in configuration 300C, which provides for a shorter vertical distance between chin cup 112 and the fixed point of collar body 120 than configurations 300A and 300B. In configuration 300C, left end member 132C is disposed partially or fully within sixth notch 136C and right end member 132F is disposed partially or fully within second notch 134C, which is horizontally aligned with sixth notch 136C.

In FIG. 3D, brace 100 is in configuration 300D, which provides for a shorter vertical distance between chin cup 112 and the fixed point of collar body 120 than configurations 300A, 300B and 300C. In configuration 300D, left end member 132C is disposed partially or fully within sixth notch 136D and right end member 132F is disposed partially or fully within second notch 134D, which is horizontally aligned with sixth notch 136D.

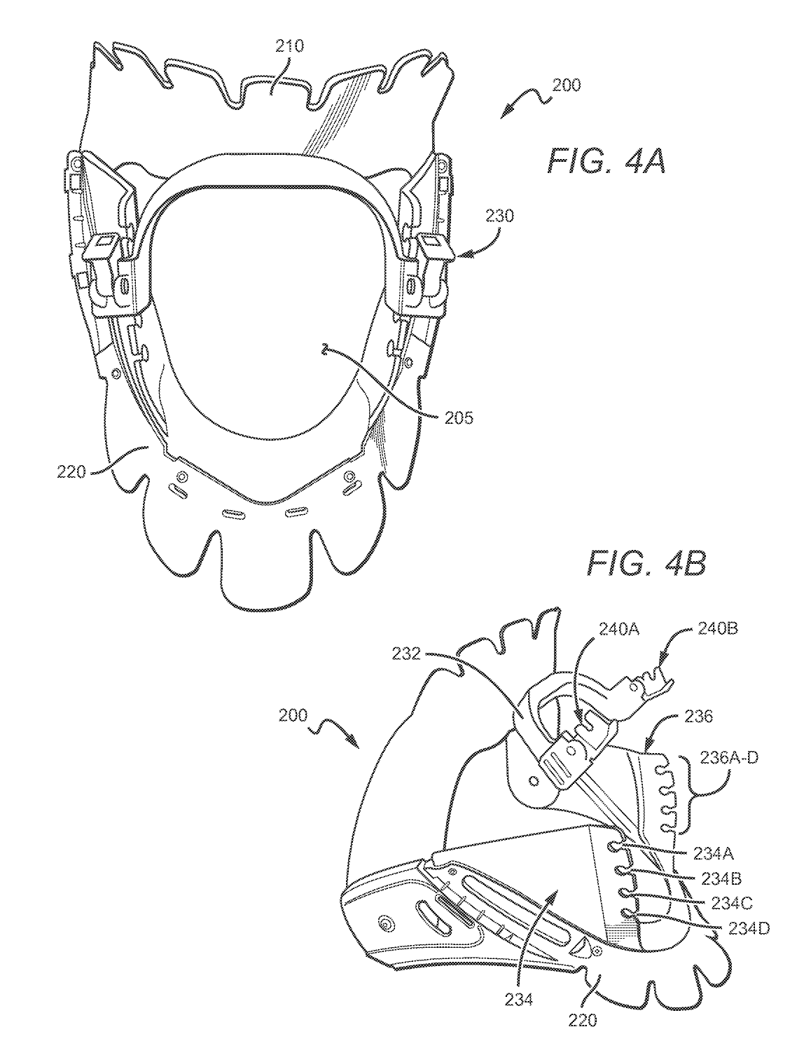

Some contemplated collars of the inventive subject matter include members that have thicker inner portions and thinner outer portions (or vice versa), such that the members can be squeezed towards one another (or pulled away from each other) and removed from notches via the thinner outer portions. FIGS. 4A-4B show front and side perspective views of an alternative brace 200, which includes such members.

Brace 200 includes a chin support 210 coupled to collar body 220 via an adjustment mechanism 230. Adjustment mechanism 230 allows a user to modify the size of tracheal opening, and includes a U-shaped adjustment piece 232 having two adjustment ends, locking members 240A and 240B, a first notched member 234 including first, second, third and fourth notches 234A-234D, and a second notched member 236 including fifth, sixth, seventh and eight notches 236A-236D.

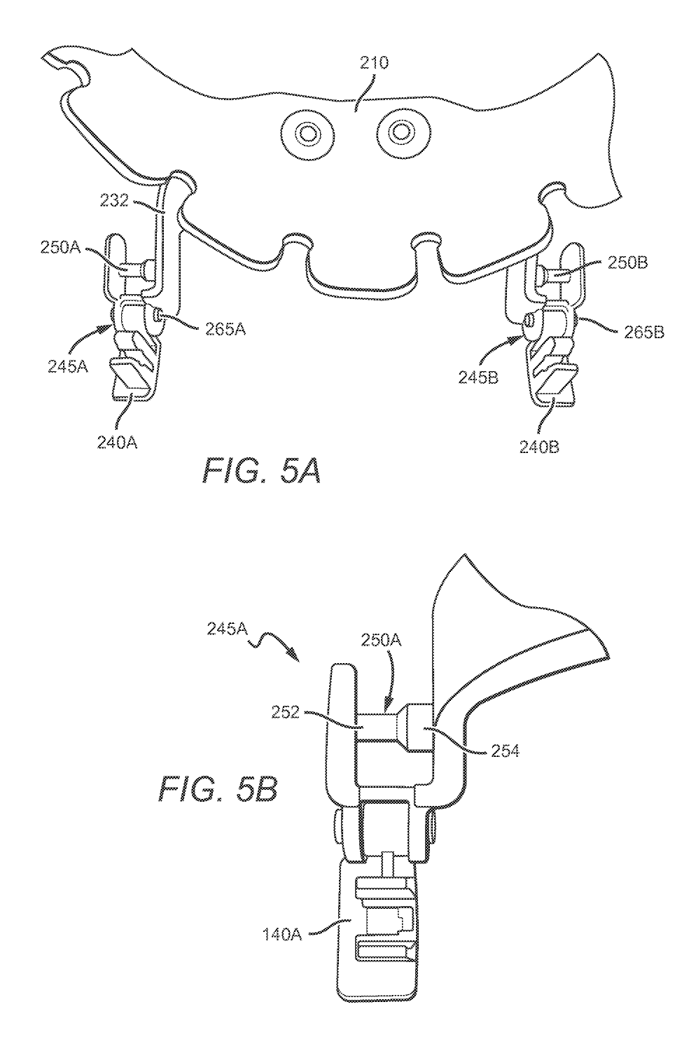

FIGS. 5A-5B provide more detailed views of adjustment piece 232's first and second adjustment ends. The interior surface of chin support 210 (the portion that would face a wearer's chin) is shown, including two fasteners through which first adjustment end 245A and second adjustment end 245B are coupled. As illustrated, adjustment ends 245A and 245B are part of a single adjustment piece 232 that is centrally attached to chin support 210. However, in some embodiments, two adjustment ends of the inventive subject matter could comprise two separate pieces that are separately coupled to a chin support.

First adjustment end 245A includes a member 250A disposed between two parallel walls. End 245A further comprises a locking member 240A, which is movably coupled to the rest of adjustment end 245A via hinge 265A or other pivoting point. Second adjustment end 245B includes member 250B disposed between two parallel walls, and locking member 240B movably coupled to the rest of end 245B via hinge 265B or other pivoting point. Member 250A of first end 245A includes an outer portion 252, further in position from member 250B, which is thinner than inner portion 254, closer in position to member 250B. Member 250B similarly includes an outer portion, further in position from member 250A, which is thinner than an inner portion, closer in position to member 250A.

As further described below, locking member 240A includes two prongs that form a gap positioned, sized and dimensioned to receive first portion 252 when second portion 254 is at least partially seated in a notch of a collar body's notched member.

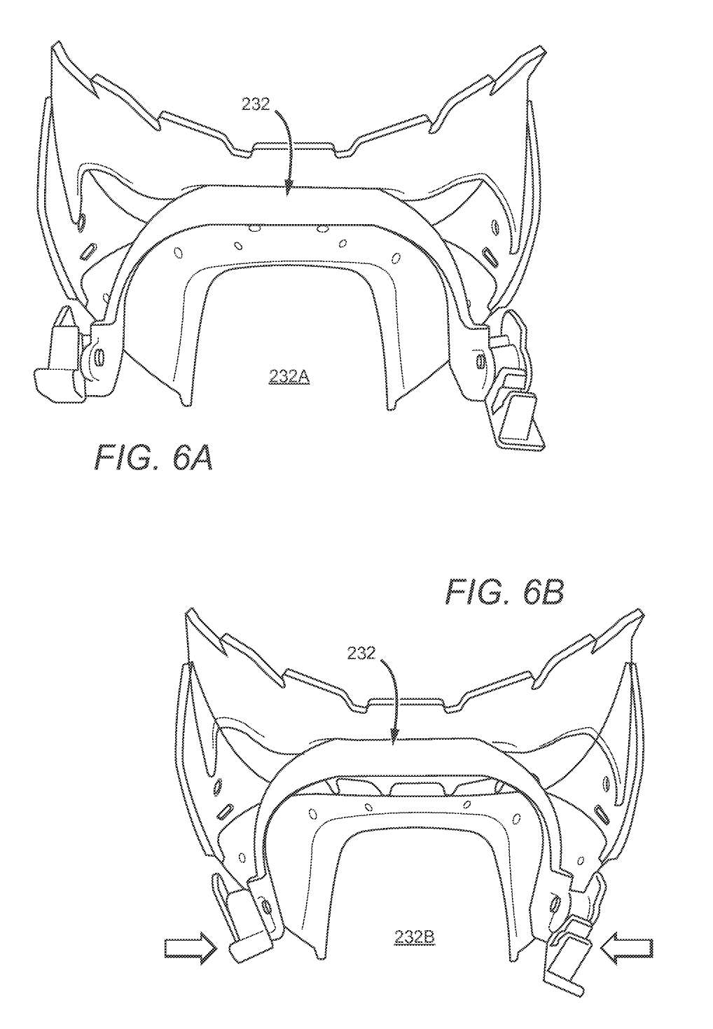

FIGS. 6A-6B show a manipulation of adjustment piece 232 between first configuration 232A and second configuration 232B. The adjustment ends of adjustment piece 232 are biased to be in the first configuration 232A, wherein the inner portion of each of members 250A and 250B could be positioned within recessed portions of corresponding notches. The adjustment ends of adjustment piece 232 could be squeezed together to be in second configuration 232B, such that the outer portion of each of members 250A and 250B could be positioned within recessed portions of a pair of notches, and moved out of and into one or more pairs of notches via the outer edges of the notches. Once the user releases the adjustment ends of adjustment piece 232, the adjustment piece would return to first configuration 232A.

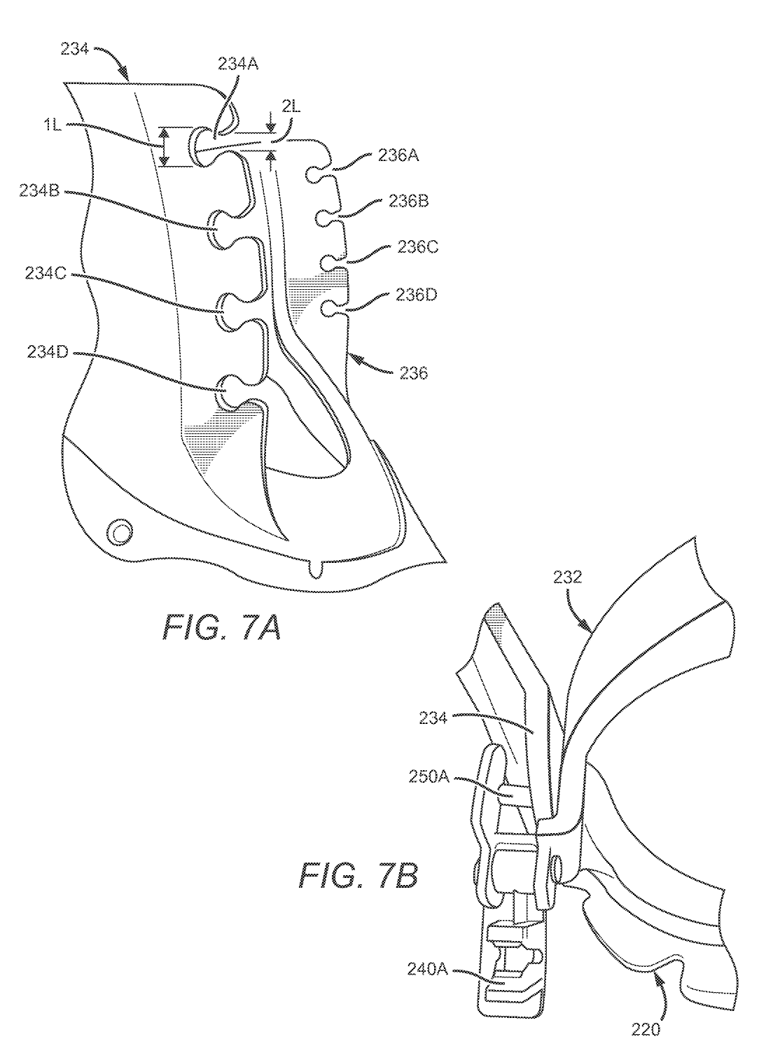

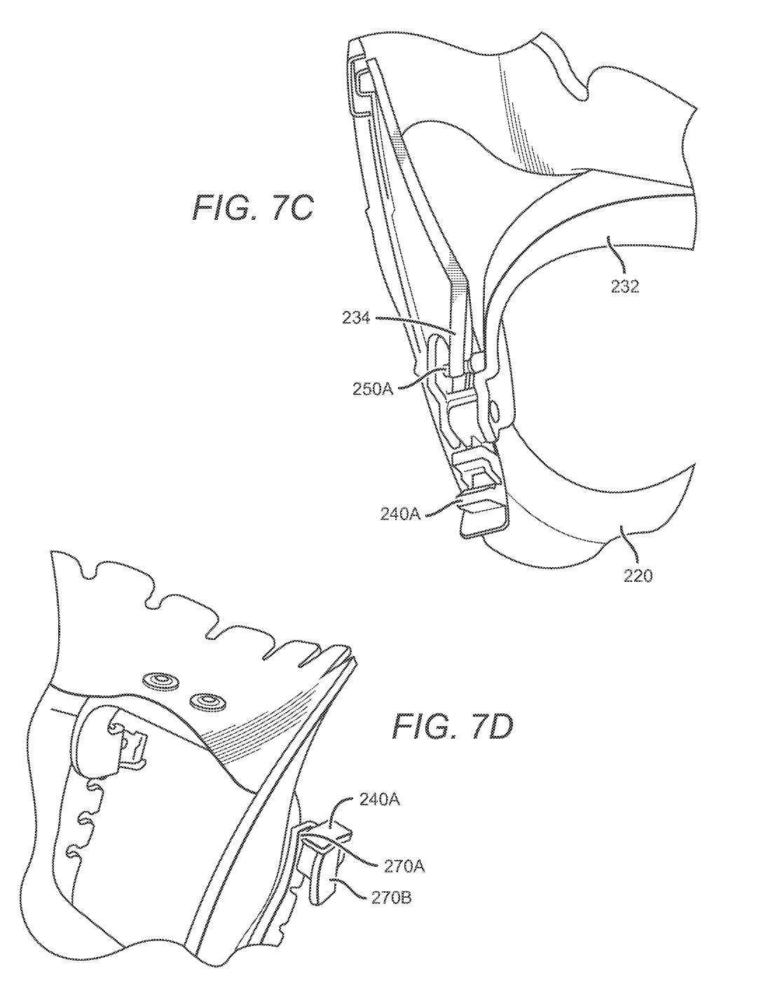

FIG. 7A illustrates first and second notched members 234 and 236 of a collar body 220 that is adjustably coupled to chin support 210. First notched member 234 includes first, second, third and fourth notches, 234A, 234B, 234C and 234D, respectively, each of which extend orthogonally to the length of member 250A. Second notched member 236 includes fifth, sixth, seventh and eight notches, 236A, 236B, 236C and 236D, respectively, each of which extend orthogonally to the length of member 250B. In some preferred embodiments, first and second notched members are vertically oriented and parallel to one another.

Some or all of the notches have a recessed portion having first length 1L, and an outer edge portion having second length 2L, which is shorter than 1L. As used herein, an “outer edge” or “open edge” of a notch is the portion of the notch having the shortest length through which at least a portion of a corresponding member could pass.

When adjustment piece 232 having adjustment ends 245A and 245B is in first configuration 232A, inner portion 254 of first member 250A could be at least partially disposed within one of notches 234A, 234B, 234C and 234D, and the inner portion of second member 250B could be at least partially disposed within one of notches 236A, 236B, 236C and 236D. Because the outer edges of the notches can have opening lengths that are smaller than the thicknesses of the inner portions (e.g., 254), it is contemplated that once members 250A and 250B are positioned within recessed portions of a pair of notches, for example notches 234A and 236A, members 250A and 250B may not be removable from within the notches until an adjustment is made (without breakage or damage to the brace).

To move from notches 234A and 236A to another pair of notches, for example, notches 234C and 236C, a user could squeeze the adjustment ends together as shown in FIG. 6B. When the adjustment piece is in configuration 232B, the thinner outer portion 242 of first member 250A, and the thinner outer portion of second member 250B could be aligned with the notches 234A and 236A, such that members 250A and 250B could be removed through the outer edges and optionally be inserted through outer edges of another pair of notches. Once adjustment ends are released and in configuration 232A, the inner portions of the members could once again be positioned within a pair of notches, and not removable until an adjustment is made.

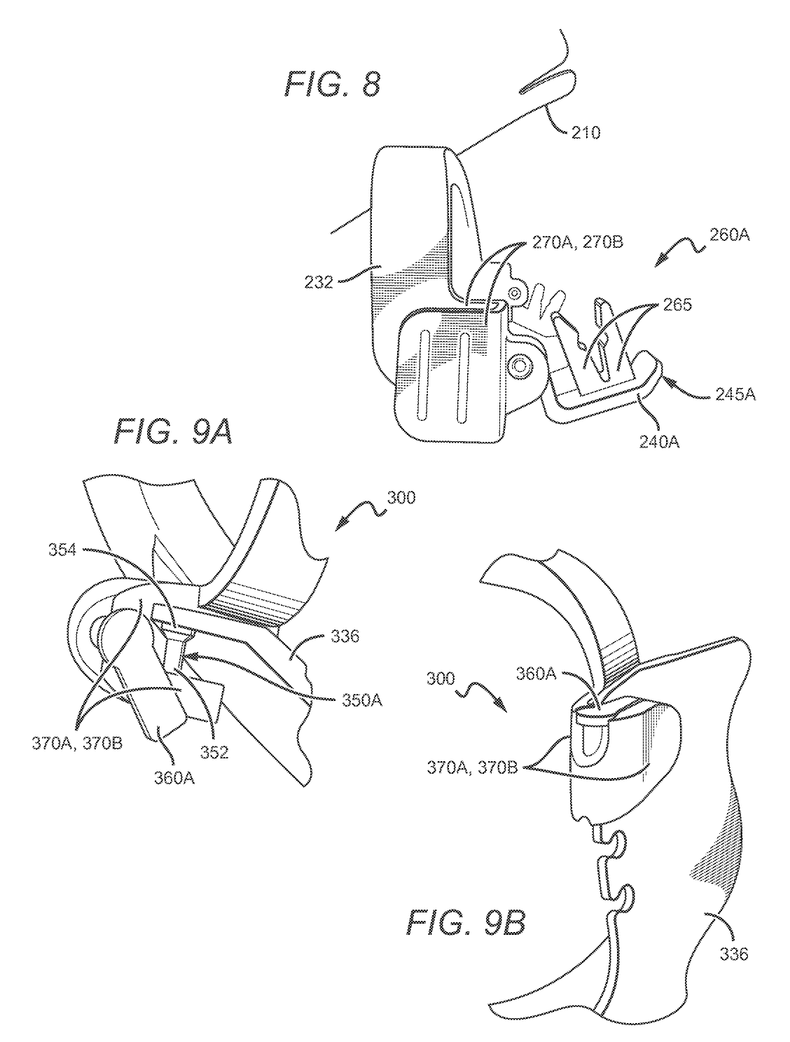

FIG. 7B illustrates member 250A disposed within first notch 234A of collar body 220's notched member 234 when adjustment end 245A of adjustment piece 232 is in configuration 232A. As locking member 240A is in an open configuration, it can be seen that outer portion 252 of member 250A is exposed, and some or all of thicker inner portion 254 is seated within first notch 234A. The locking member 240A, shown in greater detail in FIG. 7D (locked configuration) and FIG. 8 (open configuration), includes a pair of prongs 265 that define an opening aligned with, and sized and dimensioned to receive, outer portion 252. The pair of prongs 265 are part of a locking piece that is coupled to the rest of adjustment end 245A via a hinge or other pivoting mechanism. The locking piece is configured to rotate above parallel walls 270A and 270B, and prongs 265 are sized and dimensioned to fit in between parallel walls 270A and 270B.

When adjustment end 245A is in configuration 232A, locking member 240A could swing around and above the top of first and second parallel walls 270A and 270B of adjustment end 245A such that at least a portion of outer portion 252 is received by the opening defined by prongs 265, and at least a portion of prongs 265 are disposed between first and second walls 270A and 270B. This allows a user to securely wear the adjustable collar with a desired vertical length between the chin support and the collar body without worrying about unintentional adjustments being made in a vertical direction or a forward/backward direction. Viewed from another perspective, when the collar is locked in a desired position, a portion of a notched member, and at least portions of a pair of prongs is disposed between parallel walls on each adjustment end, with a member extending through the pair of prongs and the recessed portion of a notch between the parallel walls. Although not required, member 250B could also be in the configuration shown in 232A. Alternatively, member 250B could be squeezed towards member 250A and in the configuration shown in 232B.

While the discussion herein is generally directed to locking members that are rotated (at least partially) about the horizontal axis, it should be appreciated that contemplated locking members could be coupled to adjustment ends in any suitable manner, and configured to be adjusted from an open to locked position in any suitable manner. Viewed from another perspective, a locking member could be coupled with an adjustment end such that the locking member is rotatable about the horizontal axis, the vertical axis, a diagonal line, or any combination thereof.

FIG. 7C illustrates member 250A disposed within first notch 234A of collar body 220's notched member 234 when adjustment end 245A of adjustment piece 232 is in the configuration shown in 232B. Locking member 240A is permanently aligned with outer portion 252, and is also aligned with first notched member 234A while in configuration 232B. Therefore, the locking member cannot be moved to the locked configuration, shown in FIG. 7D. Although not required, member 250B could also be in the configuration shown in 232B. Alternatively, member 250B could be left in its biased position and not removable from a corresponding notch until an adjustment is made.

It is also contemplated that in some embodiments, the outer portions of members could be thicker than the inner portions, and the adjustment ends could be biased to have the thicker outer portions align with the notched members. The locking member prongs could be positioned, sized and dimensioned to receive the thinner inner portions. Where an adjustment of the vertical distance between the chin support and collar body is desired, a user could apply a force to separate the adjustment ends further away from one another such that the thinner inner portions are aligned with the notches.

Additionally or alternatively, it is contemplated that in some embodiments, one adjustment end could include a member having a thinner outer portion, and another adjustment end could include a member having a thickner outer portion. Again, the adjustment ends could be biased to have the thicker outer portions align with the notched members. Where an adjustment of the vertical distance between the chin support and collar body is desired, a user could move the adjustment ends in the same direction such that the thinner inner portions are aligned with the notches.

Additionally or alternatively, it is contemplated that in some embodiments, one or more of the adjustment ends could include a member having a thicker central portion disposed between thinner inner and outer portions. In such embodiments, the adjustment end could include or be configured to receive a locking member that includes two separate pairs of prongs. The first pair of prongs could be positioned, sized and dimensioned to receive the inner portion, and the second pair of prongs could be positioned, sized and dimensioned to receive the outer portion. A portion of the notched member and the thicker central portion of the member could be disposed between the two pairs of prongs when the locking member is in a locked position.

FIGS. 9A-9B show yet another alternative adjustment end of the inventive subject matter. Similarly to the braces described above, brace 300 includes a notched member 336 to which an adjustment end can be locked in place. The adjustment end includes a curved piece of material including parallel walls 370A and 370B. Member 350A is disposed between parallel walls 370A and 370B, and includes a first thinner portion 352, and a second thicker portion 354. Here, locking member 360A is a removable piece that includes a top base and a projection, wherein the projection includes a recess sized and dimensioned to receive a portion of the member 350A. When the projection is inserted between the parallel walls, it is contemplated that the projection will receive the portion of member 350A and tightly fit between the notched member 336 and a wall. Viewed from another perspective, a user will need to apply a downward force to the locking member 360A in order to lock an adjustment end in place. The curved piece of material could include a recess at the top portion, which enables a user to remove the locking member 360A, which is in some preferred embodiments, flush with the parallel walls (from the top, and optionally the bottom) when used with brace 300.

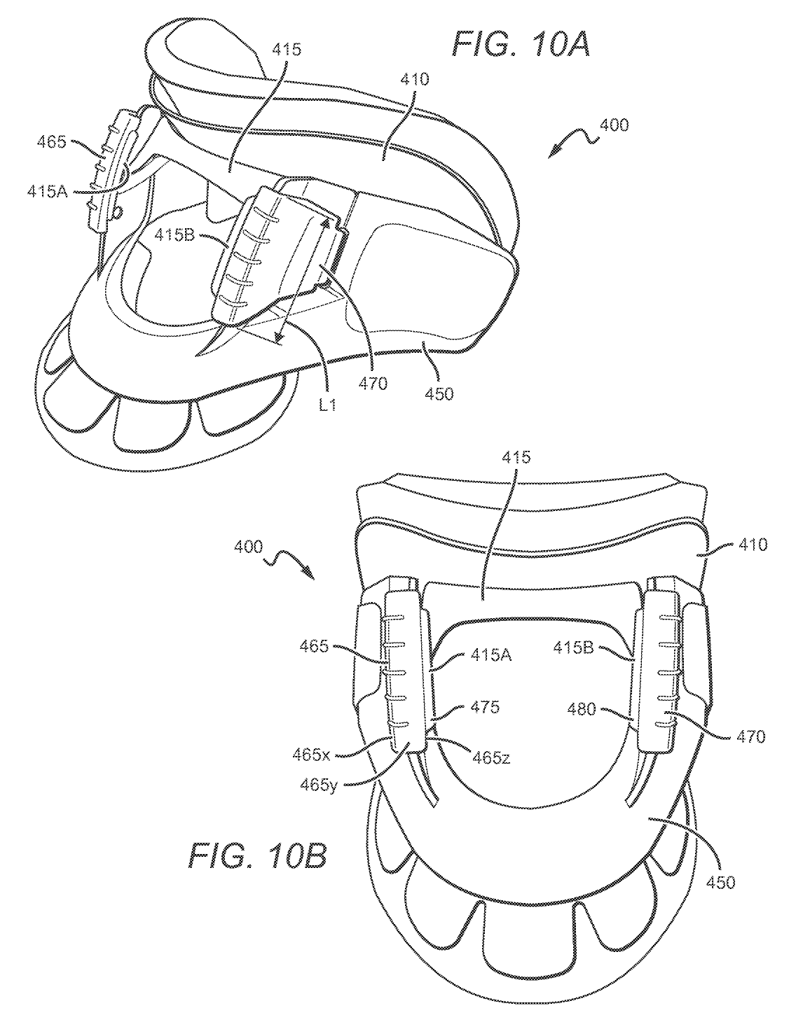

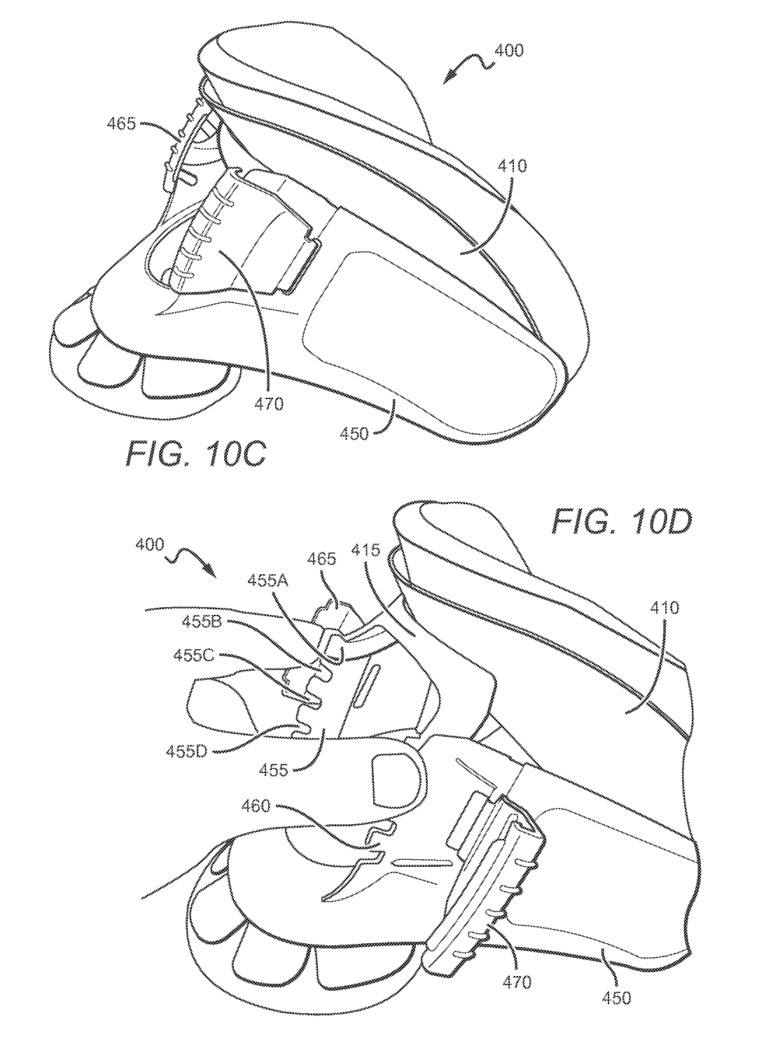

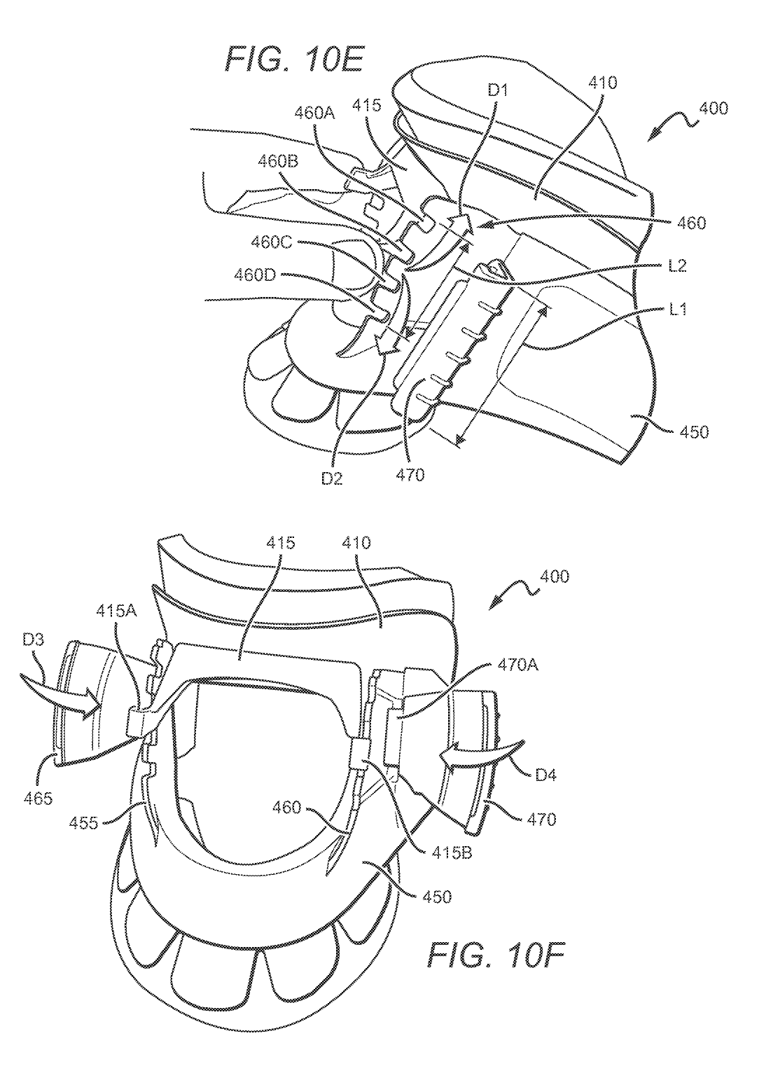

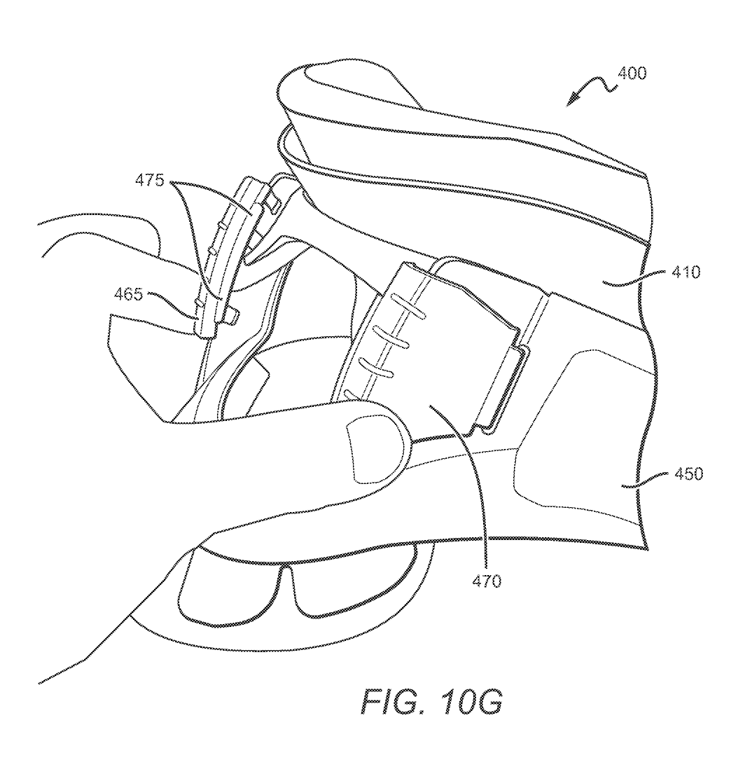

FIGS. 10A-10G illustrate another adjustable collar having a chin support adjustably coupled to a collar body. The chin support could comprise any commercially suitable chin support, including any of the chin supports described above. The collar body could comprise any commercially suitable collar body, including any of the collar bodies described above.

Collar 400 advantageously includes two locking doors or panels 465 and 470 that are configured to securely lock two adjustment ends in place regardless of which pair of notches the adjustment ends are positioned within.

Chin support 410 is pivotally or otherwise adjustably coupled to collar body 450 at right and left ends, and an adjustment piece 415 having right adjustment end 415A and left adjustment end 415B is coupled to and extends below chin support 410. It is contemplated that a user could apply a force to a single point of adjustment piece 415 to simultaneously adjust right adjustment end 415A and left adjustment end 415B. Some contemplated adjustment pieces include a vertically oriented right portion coupled to a right member, a vertically oriented left portion coupled to a left member, and a central portion extending between the right and left portions. In some alternatively embodiments, right and left vertically oriented portions could be separately coupled to (or extend from) a chin piece.

In the embodiment shown in FIGS. 10A-10G, each of the adjustment ends include a pair of parallel walls and a member extending there-between. It should be appreciated, however, that collar 400 could include any commercially suitable adjustment end, including any of the adjustment ends or members described above. For example, an adjustment end could comprise any piece(s) of material having a projection that can be at least partially and securely received by a corresponding notch.

Collar body 450 could comprise or be coupled to right notched member 455 having vertically arranged notches 455A-D, and left notched member 460 having vertically arranged notches 460A-D. Each of notches 455A-D could be sized and dimensioned to receive at least a portion of right adjustment end 415A's member, and each of notches 460A-D could be sized and dimensioned to receive at least a portion of left adjustment end 415B's member. In some preferred embodiments, the notched members are vertically oriented, the notched portions of the notched members are parallel to one another, the right and left members extend orthogonally to each of the notches, and each of the notches have the same shape and size.

Adjustment ends 415A and 415B can advantageously be adjusted between any and all of the first pair of notches (455A and 460A), the second pair of notches (455B and 460B), the third pair of notches (455C and 460C), and the fourth pair of notches (455D and 460D) in any suitable direction, including for example, directions D1 (upwards and backwards) or D2 (downwards and forwards). Such adjustments could adjust a vertical position of chin support 410 relative to collar body 450. As one non-limiting example, to increase a size of a tracheal opening defined by chin support 410 and collar body 450, adjustment ends 415A and 415B could be moved forward (in a direction away from a wearer when the brace is worn) from within the fourth pair of notches 455D and 460D, moved upward and backward in direction D1, and released such that the adjustment ends move backwards into lockable configurations and are positioned within the first pair of notches 455A and 460A (or any other pair of notches).

As illustrated, notches 455A-D and 460A-D comprise open outer edges and recessed portions. However, it should be appreciated that in some contemplated embodiments, the notches could comprise through-holes (holes that are completely bordered by the material of the notched member), and the adjustment ends could comprise protrusion members that extend horizontally from a base portion of the adjustment end and orthogonally to the notched member. To increase or decrease the size of a tracheal opening defined by a chin support and collar body, the adjustment ends could be moved in a sideways direction (left or right relative to a wearer when the brace is worn) such that the entire protrusion member is positioned on a side of the notched member. The end could then be moved in direction D1 or D2, and released such that the protrusion member is positioned through a selected notch of the notched member.

In some embodiments, adjustment ends 415A and 415B could be biased to be in a lockable configuration (with members of the adjustment ends positioned within a pair of notches, members at least partially received by a pair of notches). A user could move the adjustment ends to a movable configuration as shown in FIG. 10E (wherein the adjustment ends are movable in directions D1 or D2, or vertically, outside and in front of (or to a side of) notches), and the adjustment ends could be released back into the biased lockable configuration at the same pair of notches or a different pair of notches.

When adjustment ends 415A and 415B are positioned within a pair of notches at a desired height, a user could move right locking panel 465's receiving end in direction D3 over and around a portion of adjustment end 415A, and move left locking panel 470's receiving end in direction D4 over and around a portion of adjustment end 415B. In some embodiments, it is contemplated that when adjustment ends 415A and 415B are in a movable configuration as shown in FIG. 10E, locking panels 465 and 470 would not be able extend over adjustment ends 415A and 415B, respectively, because the adjustment ends could extend further out in a forward (or a sideways) direction than the panels could reach.

Right panel 465 could be movably coupled to the right side of collar body 450 (or to right notched member 455), and left panel 470 could be movably coupled to the left side of collar body 450 (or left notched member 460) in any suitable manner. As shown in FIG. 10F, the panels can be pivotally coupled to the collar body via hinges, including hinge 470A. Alternatively or additionally, the panels can be at least one of slidably coupled, rotatably coupled, retractably coupled, extendably coupled, and removably coupled to the collar body, notched member(s), or any other suitable portions of the brace.

The length of a panel (L1 of FIG. 10A) at its receiving end could be greater than the length of the portion of a notched member that includes a plurality of notches (L2 in FIG. 10E). Viewed from another perspective, the length of the receiving end of a panel could be greater than the length of the portion of a notched member that includes 2, 3, 4, 5 or even more notches. As such, the panel could be configured lock an adjustment end in place when the adjustment end is positioned within any notch of two or more different notches.

To lock an adjustment end in place (block its member from moving forward and out of a notch, and block the adjustment end from moving out of a locked configuration), a user could move a panel's receiving end towards the adjustment end and cause the panel's receiving end to wrap around at least a portion of the adjustment end. Here, right panel 465 has a receiving end that includes an outer wall 465X, front wall 465Y, and inner wall 465Z. The receiving end can be sized and dimensioned to wrap around a portion of the adjustment end and a corresponding notched member.

The inner wall 465Z can be long (vertically) and relatively narrow (horizontally). For example, the inner wall could have a width of less than 20 mm, less than 15 mm, less than 10 mm, or even less than 5 mm. Additionally or alternatively, the inner wall could have a width that is less than 20%, less than 15%, less than 10%, or even less than 5% of a width of the outer wall 465X. Additionally or alternatively, the distance between the outer and inner walls could be substantially the same as the thickness of an adjustment end. For example, the distance between the outer and inner walls could be within 15%, within 10%, or even within 5% of the thickness of the adjustment end. Additionally or alternatively, the width of the inner wall could be greater than the difference between (1) the distance between outer and inner walls, and (2) the thickness of the adjustment end. While the outer wall 465X is illustrated as being wide relative to inner wall 465Z. it should be appreciated that a panel of the inventive subject matter could comprise an outer wall that has a width of any suitable size (e.g., less than 20 mm, less than 15 mm, less than 10 mm, less than 5 mm, between 10 mm and 500 mm, between 10 mm and 200 mm, between 10 mm and 100 mm, between 25 mm and 75 mm). Additionally or alternatively, the outer wall could have a width that is less than, equal to, or greater than the width of the inner wall. For example, the outer wall could have a width that is between 500-2000% greater than, between 10-1000% greater than, between 100-1000% greater than, between 300-800% greater than, or even less than the width of the inner wall.

To release the adjustment end, a user could use a release tab (e.g., right release tab 475 for right panel 465, left release tab 480 for left panel 470), to cause inner wall (e.g., 465Z) to extend away from a surface or ridge of the adjustment end so that the user could release the adjustment end from the panel's receiving end. In some preferred embodiments, the release tab of a panel could extend orthogonally to the panel's inner wall.

It should be apparent to those skilled in the art that many more modifications besides those already described are possible without departing from the inventive concepts herein. The inventive subject matter, therefore, is not to be restricted except in the spirit of the appended claims.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. An adjustable collar comprising:
a chin support coupled to a collar body;
an adjustment mechanism configured to enable an adjustment of a vertical position of the chin support relative to the collar body;
a first locking panel pivotally coupled to the collar body;
wherein the adjustment mechanism comprises:
first and second adjustment ends extending below the chin support, wherein the first adjustment end comprises a first member; and
first and second notched member located on the collar body, wherein the first notched member comprises a first plurality of notches, each of which is sized and dimensioned to receive at least a portion of the first member; and
wherein the first locking panel is configured to block a movement of the first member when the first member is positioned within any notch of the first plurality of notches.

2. The adjustable collar of claim 1, wherein the second adjustment end comprises a second member, and wherein the second notched member comprises a second plurality of notches, each of which is sized and dimensioned to receive at least a portion of the second member.

3. The adjustable collar of claim 2, further comprising a second locking panel movably coupled to the collar body, and configured to block a movement of the second member when the second member is positioned within any notch of the second plurality of notches.

4. The adjustable collar of claim 1, wherein the first locking panel includes a receiving end having a greater length than a length of a portion of the first notched member that includes the first plurality of notches.

5. The adjustable collar of claim 1, wherein the first plurality of notches comprises at least 3 notches.

6. The adjustable collar of claim 2, further comprising an adjustment piece coupled to the chin piece, and wherein the adjustment piece comprises the first and second adjustment ends.

7. The adjustable collar of claim 6, wherein a manipulation of the adjustment piece simultaneously allows movement of the first and second members from a first set of notches to a second set of notches, respectively, and wherein each of the first and second sets of notches includes a notch from the first plurality of notches and a notch from the second plurality of notches.

8. The adjustable collar of claim 6, wherein the adjustment piece is biased in a lockable configuration such that the first member is positioned within a notch of the first plurality of notches, and wherein the second member is positioned within a notch of the second plurality of notches.

9. The adjustable collar of claim 1, wherein the first locking panel is sized and dimensioned to wrap around an outer edge of the first notched member and be in a locked position when the first member is positioned within a notch from the first plurality of notches.

10. The adjustable collar of claim 9, wherein the first locking panel includes a release tab that can be utilized to release the first locking panel from the locked position.

11. The adjustable collar of claim 1, wherein the first adjustment end comprises first and second parallel walls, and wherein the first member is disposed between the first and second parallel walls.

12. The adjustable collar of claim 1, wherein the first member comprises a protrusion extending from a base portion of the first adjustment end, and wherein the protrusion is sized and dimensioned to be securely received by at least one of the first plurality of notches.

13. The adjustable collar of claim 9, further comprising a second locking panel, wherein the second locking panel is sized and dimensioned to wrap around an outer edge of the second notched member and be in a locked position when the second member is positioned within a notch from the second plurality of notches.

14. An adjustable collar comprising:
a chin support coupled to a collar body, the chin support comprising a chin cup;
an adjustment piece extending below the chin cup, the adjustment piece including a central portion that extends between right and left members;
a right notched member coupled to the collar body and comprising a first plurality of notches, each of which is sized and dimensioned to receive at least a portion of the right member; and
a left notched member coupled to the collar body and comprising a second plurality of notches, each of which is sized and dimensioned to receive at least a portion of the left member.

15. The adjustable collar of claim 14, further comprising a right locking panel movably coupled to a right side of the collar body, and configured to block a movement of the right member when the right member is positioned within any notch of the first plurality of notches.

16. The adjustable collar of claim 15, further comprising a left locking panel movably coupled to a left side of the collar body, and configured to block a movement of the left member when the left member is positioned within any notch of the second plurality of notches.

17. The adjustable collar of claim 14, wherein the adjustment piece comprises a vertically oriented right portion coupled to the right member, and a vertically oriented left portion coupled to the left member.

18. The adjustable collar of claim 13, wherein the adjustment piece is biased to be in a lockable configuration such that the right member is positioned within a notch of the first plurality of notches, and the left member is positioned within a notch of the second plurality of notches.

19. The adjustable collar of claim 18, wherein the right locking panel is sized and dimensioned to wrap around an outer edge of the right notched member when in a locked position.

20. The adjustable collar of claim 19, wherein the right locking panel is in the locked position only when the adjustment piece is in the lockable configuration.

21. The adjustable collar of claim 20, wherein the adjustment piece can be pulled out of the lockable configuration for adjustment of a vertical position of the chin piece relative to the collar body when the right locking panel is in an unlocked position.
